(19) United States
(12) Patent Application Publication
(10) Pub. No.: US 2025/0098950 A1
(43) Pub. Date: Mar. 27, 2025

(54) PROCESSOR FOR ENDOSCOPE AND METHOD OF CONTROLLING PROCESSOR FOR ENDOSCOPE

(71) Applicant: HOYA CORPORATION, Tokyo (JP)

(72) Inventor: Yoshihiro HAYASHI, Tokyo (JP)

(73) Assignee: HOYA CORPORATION, Tokyo (JP)

(21) Appl. No.: 18/833,571

(22) PCT Filed: Apr. 20, 2023
(86) PCT No.: PCT/JP2023/015794

§ 371 Date: Jul. 26, 2024

(30) Foreign Application Priority Data
May 10, 2022 (JP) 2022-077751

(51) Int. Cl. A61B 1/06; A61B 1/045

(52) U.S. Cl. CPC A61B 1/0655; A61B 1/045; A61B 1/0638

(57) ABSTRACT

A processor for an endoscope, includes: an imaging signal acquisition unit that acquires an imaging signal from an image sensor; and a dimming unit that controls a light source that illuminates an observation visual field based on the imaging signal, in which the dimming unit includes: a luminance index acquisition unit that acquires, based on the imaging signal, a first index related to a luminance of a first color and a second index related to a luminance of a second color; a generation unit that generates a photometric signal based on the first index and the second index; and a dimming output unit that outputs a dimming signal to the light source based on the photometric signal.

### TECHNICAL FIELD

The present invention relates to a processor for an endoscope and a method of controlling the processor for an endoscope.

### BACKGROUND ART

A processor for an endoscope having an automatic dimming function for automatically adjusting the brightness of illumination light based on the brightness of an endoscope image, is used. There is proposed a processor for an endoscope that reduces halation by automatically adjusting a dimming mode when the halation occurs in an endoscope image (Patent Literature 1).

### CITATION LIST

### Patent Literature

Patent Literature 1: WO 2017/126531 A

### SUMMARY OF INVENTION

### Technical Problem

Even when a doctor operates an endoscope to change the visual field, the automatic dimming function maintains an endoscope image at brightness suitable for diagnosis.

However, when a distal end of the endoscope is close to or in contact with a biological tissue such as a mucous membrane, sufficient illumination light does not reach to the visual field of the endoscope. Therefore, the endoscope image becomes dark. In such a situation, even when the illumination light is brightened, the endoscope image cannot have a predetermined brightness. Therefore, the automatic dimming function does not operate normally.

In one aspect, an object is to provide a processor for an endoscope in which an automatic dimming function appropriately operates even when a distal end of an endoscope is close to or in contact with a biological tissue.

### Solution to Problem

A processor for an endoscope, includes: an imaging signal acquisition unit that acquires an imaging signal from an image sensor; and a dimming unit that controls a light source that illuminates an observation visual field based on the imaging signal, in which the dimming unit includes: a luminance index acquisition unit that acquires, based on the imaging signal, a first index related to a luminance of a first color and a second index related to a luminance of a second color; a generation unit that generates a photometric signal based on the first index and the second index; and a dimming output unit that outputs a dimming signal to the light source based on the photometric signal.

### Advantageous Effects of Invention

In one aspect, it is possible to provide a processor for an endoscope in which an automatic dimming function appropriately operates even when a distal end of an endoscope is close to or in contact with a biological tissue.

### BRIEF DESCRIPTION OF DRAWINGS

FIG. 1 is an explanatory diagram for explaining a configuration of an endoscope system.

FIG. 2 is an external view of an end surface of a distal end portion.

FIG. 3 is an explanatory diagram for explaining a configuration and a function of a dimming unit.

FIG. 4 is a diagram for explaining an influence of a distance between the distal end portion and a biological tissue on an imaging signal.

FIG. 5 is a graph for explaining the influence of the distance between the distal end portion and the biological tissue on the imaging signal.

FIG. 6 is a graph for explaining a coefficient K.

FIG. 7 is a flowchart for explaining a flow of operation of the dimming unit.

FIG. 8A is an explanatory diagram for explaining a contact determination method according to a second embodiment.

FIG. 8B is an explanatory diagram for explaining the contact determination method according to the second embodiment.

FIG. 9 is a flowchart for explaining a processing flow of a program according to the second embodiment.

FIG. 10 is a flowchart for explaining a processing flow of a subroutine of a contact determination.

FIG. 11 is a flowchart for explaining a processing flow of a program according to a third embodiment.

FIG. 12 is a flowchart for explaining a processing flow of a program according to a fourth embodiment.

### DESCRIPTION OF EMBODIMENTS

### First Embodiment

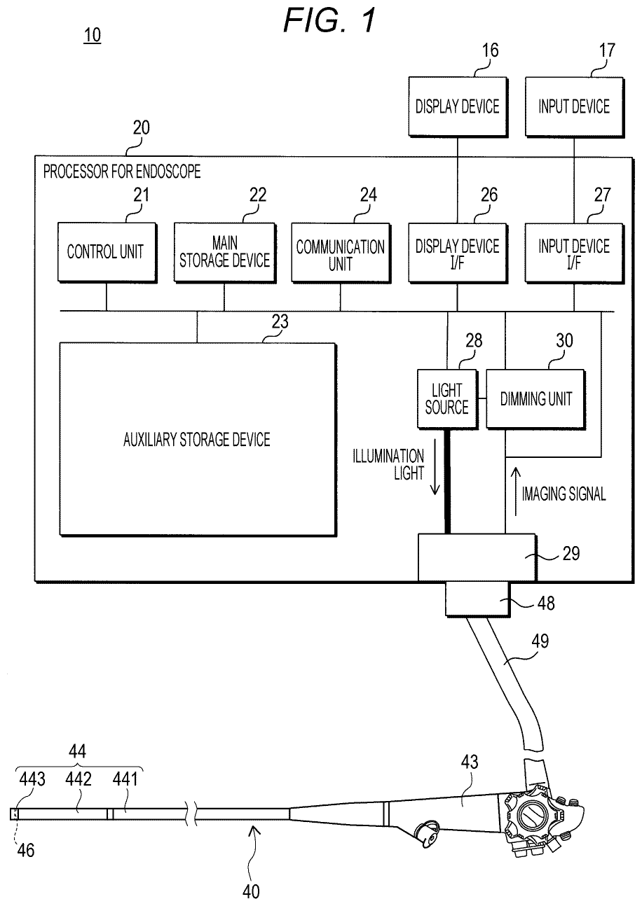

FIG. 1 is an explanatory diagram for explaining a configuration of an endoscope system 10. The endoscope system 10 includes a processor 20 for an endoscope, an endoscope 40, a display device 16, and an input device 17. The processor 20 for an endoscope includes a control unit 21, a main storage device 22, an auxiliary storage device 23, a communication unit 24, a display device interface (I/F) 26, an input device I/F 27, a light source 28, a dimming unit 30, a connector 29 for an endoscope, and a bus.

The endoscope 40 includes an insertion portion 44, an operation unit 43, a universal cord 49, and a scope connector 48. The insertion portion 44 is long, and has one end connected to the operation unit 43 via a bend preventing portion. The insertion portion 44 includes a soft portion 441, a bending portion 442, and a distal end portion 443 in this order from the operation unit 43 side. The bending portion 442 is bent according to the operation of a bending knob provided in the operation unit 43.

The universal cord 49 is long, and has a first end connected to the operation unit 43 and a second end connected to the scope connector 48. The universal cord 49 is soft. The scope connector 48 has a substantially rectangular parallelepiped shape. The scope connector 48 includes a signal transmission connector and an illumination light connector. An air/water supply pump (not illustrated) is connected to the endoscope 40.

The control unit 21 is an arithmetic control device that executes a program of the present embodiment. One or a plurality of central processing units (CPUs), a multi-core CPU, or the like is used as the control unit 21. The control unit 21 is connected to each hardware unit of the processor 20 for an endoscope via the bus.

The display device 16 is, for example, a liquid crystal display device or an organic electro luminescence (EL) display device. The input device 17 is, for example, a keyboard, a touch panel, or a foot switch.

The main storage device 22 is a storage device such as a static random access memory (SRAM), a dynamic random access memory (DRAM), or a flash memory. The main storage device 22 temporarily stores information required during processing performed by the control unit 21 and a program being executed by the control unit 21.

The auxiliary storage device 23 is a storage device such as an SRAM, a flash memory, or a hard disk. The auxiliary storage device 23 stores the program to be executed by the control unit 21 and various kinds of data necessary for executing the program.

The communication unit 24 is an interface for performing data communication between the processor 20 for an endoscope and the network. The display device I/F 26 is an interface for connecting the processor 20 for an endoscope and the display device 16. The input device I/F 27 is an interface for connecting the processor 20 for an endoscope and the input device 17.

The light source 28 is a high-luminance light source such as a light emitting diode (LED). The light source 28 may be a combination of a light source that emits broadband light such as a xenon lamp, and an optical filter. The light source 28 may be an illumination LED built in the distal end portion 443.

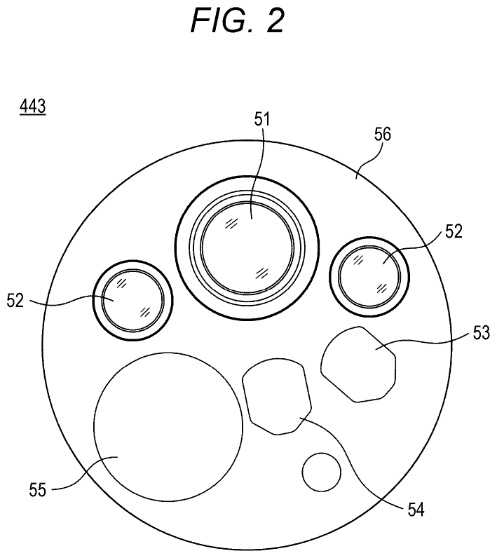

The light source 28 is connected to the bus via a driver (not illustrated). The control unit 21 controls turn-on, turn-off, brightness change, and color change of the light source 28. The illumination light emitted from the light source 28 is supplied to the endoscope 40 via the connector 29 for an endoscope and the scope connector 48. The illumination light is guided to an illumination window 52 (see FIG. 2) provided in the distal end portion 443 by an illumination optical fiber inside the endoscope 40.

FIG. 2 is an external view of an end surface of the distal end portion 443. An observation window 51, two illumination windows 52, an air supply nozzle 53, a water supply nozzle 54, a channel outlet 55, and the like are arranged at a distal end of the distal end portion 443.

Note that FIG. 2 is an example of an appearance of the end surface of the distal end portion 443, and the arrangement of each member is not limited to FIG. 2. For example, the arrangements of the air supply nozzle 53 and the water supply nozzle 54 may be reversed. The illumination window 52 may be one or three or more. The endoscope 40 may be of a side view type in which a visual field direction is set to the side of the insertion portion 44, an oblique view type, or the like.

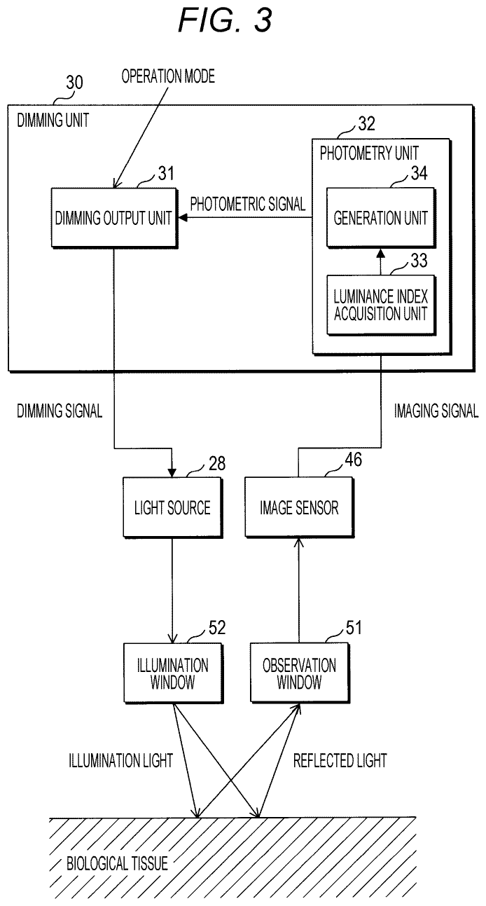

FIG. 3 is an explanatory diagram for explaining a configuration and a function of the dimming unit 30. The dimming unit 30 includes a dimming output unit 31 and a photometry unit 32. The photometry unit 32 includes a luminance index acquisition unit 33 and a generation unit 34.

The observation visual field is illuminated by the illumination light emitted from the illumination window 52. For example, the light reflected by a biological tissue in the observation visual field forms an image on an image sensor 46 via the observation window 51 and an observation optical system (not illustrated). An imaging signal generated by the image sensor 46 is transmitted to the processor 20 for an endoscope via the scope connector 48. The connector 29 for an endoscope to which the scope connector 48 is connected functions as an imaging signal acquisition unit that acquires the imaging signal from the image sensor 46.

In the following description, the endoscope system 10 using a so-called primary color image sensor 46 that outputs three color signals of red (R), green (G), and blue (B) will be described as an example. Blue is an example of a first color. Green is an example of a second color having a wavelength longer than that of the first color. Note that a complementary color image sensor 46 may be used instead of the primary color image sensor 46.

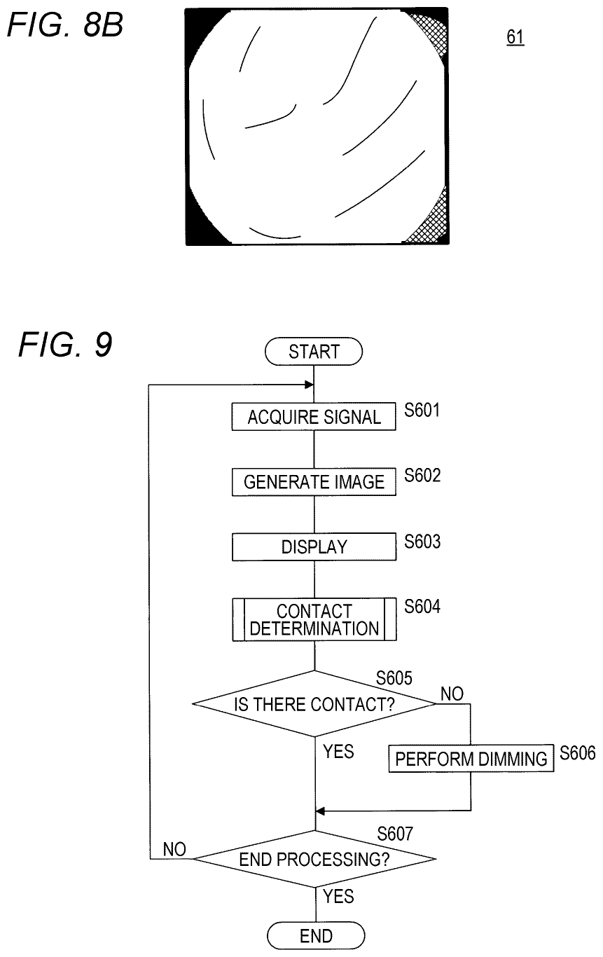

The control unit 21 performs image processing based on the imaging signal, and outputs, to the display device 16, an endoscope image 61 having image quality suitable for endoscopic examination (see FIG. 8A). The luminance index acquisition unit 33 acquires a luminance index based on the imaging signal. The luminance index is an index related to the luminance of an image captured by the image sensor 46. In the conventional dimming unit 30, an index related to the luminance of the B component of the image is typically used. Details of the luminance index will be described later.

The generation unit 34 generates a photometric signal based on the luminance index, and outputs the photometric signal to the dimming output unit 31. The dimming output unit 31 generates a dimming signal based on the photometric signal, the model of the endoscope 40, and the operation mode set by the user, and outputs the dimming signal to the light source 28. The operation modes are, for example, a normal light observation mode and a special light observation mode. The illumination light having brightness and color based on the dimming signal is emitted from the light source 28.

By looping the above flow, automatic dimming for automatically adjusting the brightness and the like of the illumination light in real time is performed. The dimming unit 30 is, for example, a custom integrated circuit (IC) such as an application specific integrated circuit (ASIC), or a programmable logic device (PLD) such as a field-programmable gate array (FPGA). The dimming unit 30 may be a dedicated IC chip designed for automatic dimming of the present embodiment.

The dimming output unit 31, the photometry unit 32, the luminance index acquisition unit 33, and the generation unit 34 included in the dimming unit 30 are logic circuits provided inside the IC. By performing dimming at a high speed using hardware, it is possible to provide the endoscope system 10 having a dimming function that quickly follows a change in the observation visual field.

The dimming unit 30 may be provided integrally with a driver IC for light source control. The dimming unit 30 may be provided on an IC chip integrated with the control unit 21. When the speeds of the control unit 21 and the bus are sufficiently high, the processing at the dimming unit 30 may be implemented by the control unit 21 by means of software.

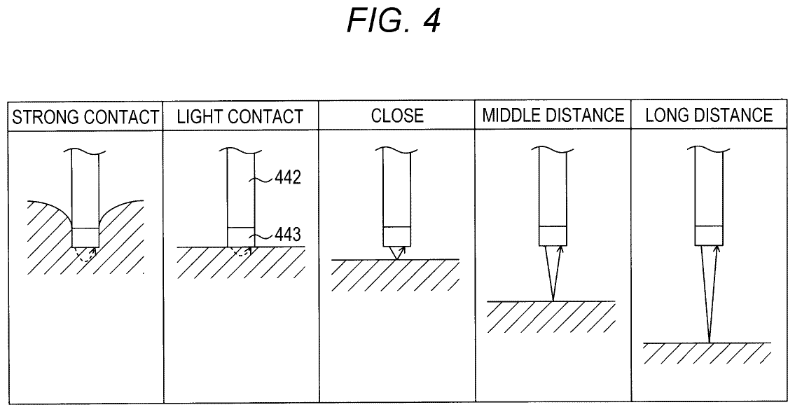

FIG. 4 is a diagram for explaining an influence of a distance between the distal end portion 443 and a biological tissue on the imaging signal. FIG. 4 schematically illustrates the influence of the distance between the distal end portion 443 and the biological tissue. The hatched portion indicates the biological tissue.

“Strong contact” is a state in which the distal end portion 443 is pressed against the biological tissue. The illumination light emitted from the illumination window 52 propagates inside the biological tissue and reaches the observation window 51. “Light contact” is a state in which the distal end portion 443 and the biological tissue are in light contact with each other, and a layer of liquid such as mucus or water is generated between the distal end portion 443 and the biological tissue due to surface tension. The illumination light emitted from the illumination window 52 propagates inside the liquid layer and through the surface layer portion of the biological tissue, and reaches the observation window 51.

In the states of “light contact” and “strong contact”, most of the energy of the illumination light is absorbed by the biological tissue, so that small amount of light reaches the observation window 51. Even when the illumination light is set to be bright, the image captured by the image sensor 46 remains dark.

“Close” is a state in which the distal end portion 443 gets closer to the biological tissue than the focusing range of the observation optical system. In the state of “close”, the endoscope image 61 is in a state of so-called “out-of-focus”. “Middle distance” is a state in which the state of the biological tissue can be observed in detail by the endoscope 40. “Long distance” is a state in which a wide range of the biological tissue can be observed by the endoscope 40. The doctor usually operates the endoscope 40 to be in the state of “long distance” or “middle distance” to perform the endoscopic examination.

In the states of “close”, “middle distance”, and “long distance”, the illumination light emitted from the illumination window 52 is reflected by the surface of the biological tissue and reaches the observation window 51. The change in the brightness of the illumination light is directly reflected on the brightness of the image captured by the image sensor 46.

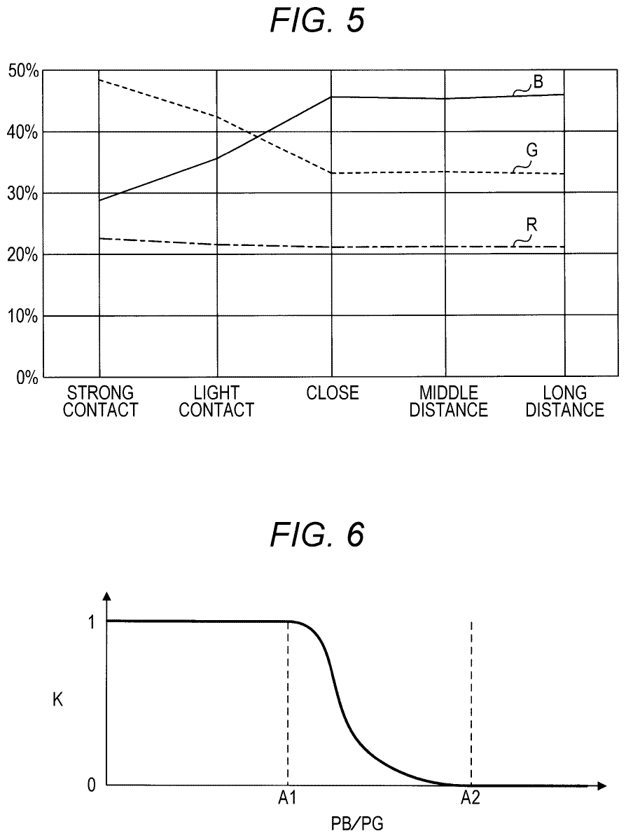

FIG. 5 is a graph for explaining the influence of the distance between the distal end portion 443 and the biological tissue on the imaging signal. The horizontal axis in FIG. 5 indicates each state described with reference to FIG. 4. The vertical axis indicates component ratios of respective colors of R, G, and B constituting the image captured by the image sensor 46. A dashed-dotted line indicates the ratio of the R component, a broken line indicates the ratio of the G component, and a solid line indicates the ratio of the B component.

In “close”, “middle distance”, and “long distance”, the component ratios are substantially constant, and the ratios of the components are larger in the order of the B component, the G component, and the R component. The ratio of the R component does not change much in “light contact” and “strong contact”. In “light contact”, the ratio of the G component is larger than that of the B component. In “strong contact”, the ratio of the G component further increases.

An outline of automatic dimming performed by the endoscope system 10 according to the present embodiment will be described. When the ratio of the B component is larger than that of the G component, the generation unit 34 generates a photometric signal based on the luminance index related to the B component. When the photometric signal indicates that an image brighter than the predetermined reference has been captured, the dimming output unit 31 outputs a dimming signal for reducing the output of the light source 28. When the photometric signal indicates that an image darker than the predetermined reference has been captured, the dimming output unit 31 outputs a dimming signal for increasing the output of the light source 28.

The distal end portion 443 is not in contact with the biological tissue, and the change in the brightness of the illumination light due to the automatic dimming is directly reflected on the brightness of the image captured by the image sensor 46. Therefore, even when the state of the observation visual field changes, the brightness of the illumination light is quickly adjusted, and the doctor can continue the endoscopic examination smoothly.

When the ratio of the G component is larger than that of the B component, the distal end portion 443 is in contact with the biological tissue. Therefore, the change in the brightness of the illumination light due to the automatic dimming is not sufficiently reflected on the brightness of the image captured by the image sensor 46. When the automatic dimming is performed based on the luminance of the B component in such a state, the automatic dimming function does not operate appropriately, and the light source 28 is continuously operated at the maximum output.

When the light source 28 is continuously operated at the maximum output for a long time, the life of the light source 28 is shortened. Since the inside of the processor 20 for an endoscope is heated due to the heat generation of the light source 28, malfunction and failure of electronic components such as the control unit 21 and the dimming unit 30 are likely to occur. There is also a problem that the power consumption of the light source 28 unnecessarily increases. Since the light emitted from the illumination window 52 is unnecessarily strong, the biological tissue may be adversely affected.

In order to prevent the occurrence of the problems described above, when the ratio of the G component is larger than that of the B component, the generation unit 34 of the present embodiment outputs a photometric signal that is large with respect to the luminance index, that is, a photometric signal similar to that in a case where the image captured by the image sensor 46 is brighter than the actual image. The dimming output unit 31 is operated based on the photometric signal. Therefore, the output of the light source 28 is not unnecessarily increased, and the occurrence of various problems described above can be prevented.

A specific example of the luminance index will be described. The luminance index is, for example, an average luminance of G and B in the entire image captured by the image sensor 46. The luminance index may be the average luminance of G and B in a predetermined region of the image captured by the image sensor 46. In the following description, the predetermined region used for calculating the luminance index may be referred to as a photometric region.

The photometric region is, for example, a central portion of the image. The photometric region may be a region near the middle between the center and the edge of the image. The control unit 21 may receive specification of a photometric region by the doctor and give an instruction to the dimming unit 30. For example, the control unit 21 may set a lesion detected by artificial intelligence (AI) as a photometric region and give an instruction to the dimming unit 30.

Instead of using the average luminance, for example, a statistical value such as a median value of the luminance may be used as the luminance index. The control unit 21 may receive specification of a method of calculating the luminance index and give an instruction to the dimming unit 30.

A specific example of the operation of the generation unit 34 that generates the photometric signal based on the luminance index will be described. The generation unit 34 outputs a photometric signal calculated based on, for example, Formula (1).

$$M = PB + K \times PG \qquad ( 1 )$$

M is a photometric signal.
PB is a luminance index related to B.
PG is a luminance index related to G.
K is a coefficient.

In Formula (1), PB is an example of a first index related to the luminance of the first color, and PG is an example of a second index related to the luminance of the second color.

FIG. 6 is a graph for explaining the coefficient K. The horizontal axis in FIG. 6 indicates a value obtained by dividing the luminance index PB related to B by the luminance index PG related to G. The vertical axis in FIG. 6 indicates the coefficient K. When PB/PG is less than a threshold A1, the coefficient K is 1. When PB/PG is greater than or equal to a threshold A2, the coefficient K is 0. In a range where PB/PG is A1 or more and less than A2, the coefficient K gradually changes from 1 to 0.

For example, the threshold A1 is 1.0, and the threshold A2 is 1.3. The region where PB/PG is less than the threshold A1 is a range where the ratio of the B component is smaller than that of the G component in FIG. 5, and is a region where the distal end portion 443 and the biological tissue are in contact with each other. The region where PB/PG is equal to or larger than the threshold A2 is a region where the ratio of the B component is 1.3 times or more the G component in FIG. 5, and is a region where the distal end portion 443 and the biological tissue are not in contact with each other.

Therefore, the photometric signal calculated based on Formula (1) is (PB+PG) when the distal end portion 443 and the biological tissue are in contact with each other, and is PB when the distal end portion 443 and the biological tissue are not in contact with each other. In a state between contact and non-contact, such as when a part of the distal end portion 443 is in contact with the biological tissue, a photometric signal between contact and non-contact is calculated.

As described above, it is possible to provide the endoscope system 10 that reduces the output of the light source 28 when the distal end portion 443 and the biological tissue are in contact with each other. Since the coefficient K gradually changes between 0 and 1 as illustrated in FIG. 6, it is possible to avoid a sudden change in the output of the light source 28 when a part of the distal end portion 443 is in contact with the biological tissue and when the contact range changes, for example. Therefore, it is possible to provide the endoscope system 10 that prevents a sudden change in the brightness of the endoscope image.

The generation unit 34 may output a photometric signal calculated based on, for example, Formula (2).

$$M = PB + K \times ( P G + PR ) \qquad ( 2 )$$

PR is a luminance index related to R.

In the case where Formula (2) is used, the photometric signal output from the generation unit 34, when the distal end portion 443 and the biological tissue are in contact with each other, is larger than that in the case where Formula (1) is used. Therefore, it is possible to provide the endoscope system 10 that further reduces the output of the light source 28 when the distal end portion 443 and the biological tissue are in contact with each other.

The generation unit 34 may output a photometric signal calculated based on, for example, Formula (3).

$$M = ( 1 - K ) P B + K \times PG \qquad ( 3 )$$

According to Formula (3), (1−K) represents the contribution ratio of the B component to a photometric signal M, K represents the contribution ratio of the G component to the photometric signal M, and the total of both the usage ratios is 100%.

The generation unit 34 may output a photometric signal calculated based on, for example, Formula (4).

$$\begin{matrix}[ Formula 1 ] & \\ M = \{ \begin{matrix}PB & ( PG < PB ) \\ PG & ( PB ≦ PG )\end{matrix} & ( 4 )\end{matrix}$$

According to Formula (4), when PB is larger than PG, the photometric signal M is a luminance index related to the B component, and when PB is equal to or less than PG, the photometric signal M is a luminance index related to the G component.) indicates that the luminance index PB related to B is used as the photometric index M when the luminance index PB is larger than the luminance index PG related to G, and the luminance index PG is used as the photometric index M when the luminance index PB is equal to or less than the luminance index PG in the graph described with reference to FIG. 5.

That is, Formula (4) indicates an operation of determining that the distal end portion 443 and the biological tissue are in contact with each other and using PG as the photometric signal M when PB is equal to or less than PG.

The generation unit 34 may output a photometric signal calculated based on, for example, Formula (5).

$$\begin{matrix}[ Formula 2 ] & \\ M = \{ \begin{matrix}PB & ( PG < PB ) \\ PG + PR & ( PB ≦ PG )\end{matrix} & ( 5 )\end{matrix}$$

Formula (5) indicates an operation of determining that the distal end portion 443 and the biological tissue are in contact with each other and using (PG+PR) as the photometric signal M when PB is equal to or less than PG.

The calculation formulas of the photometric signals exemplified by Formulas (1) to (5) are all examples, and the calculation formulas are not limited thereto. For example, the generation unit 34 may use, as the photometric signal, a value obtained by multiplying an arbitrary constant on the left side of Formulas (1) to (5). The generation unit 34 may switch the calculation formula to be used according to, for example, the model of the endoscope 40, or a condition of an operation mode in use or the like.

According to the calculation formulas of the photometric signal exemplified by Formulas (1) to (5), when PB is equal to or less than PG, there is calculated a photometric signal having a higher contribution degree of PG than when PB exceeds PG.

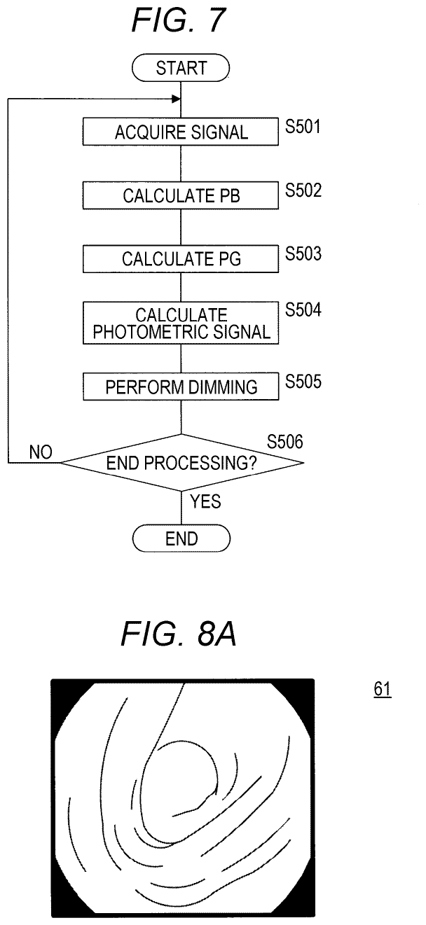

FIG. 7 is a flowchart for explaining a flow of operation of the dimming unit 30. In parallel with the processing described with reference to FIG. 7, the control unit 21 performs image processing based on the imaging signal acquired from the image sensor 46 to generate an endoscope image, and displays the endoscope image on the display device 16. For example, when a doctor operates to change the operation mode from the normal light observation mode to the special light observation mode, the control unit 21 notifies the dimming unit 30 of the change of the operation mode. The processing performed by the control unit 21, such as generation of an endoscope image, is known, and thus description thereof is omitted.

The dimming unit 30 acquires the imaging signal from the image sensor 46 (step S501). The luminance index acquisition unit 33 calculates the luminance index PB related to B based on the imaging signal (step S502). The luminance index acquisition unit 33 calculates the luminance index PG related to G based on the imaging signal (step S503). The generation unit 34 calculates the photometric signal M based on the calculation formulas exemplified by Formulas (1) to (4), for example (step S504).

The dimming output unit 31 outputs the dimming signal based on the photometric signal and the operation mode instructed from the control unit 21 (step S505). For example, when the operation mode is the normal light observation mode, the dimming output unit 31 outputs, to the light source 28, a dimming signal for emitting white light having a luminance corresponding to the photometric signal. Similarly, when the operation mode is the special light observation mode, the dimming output unit 31 outputs, to the light source 28, a dimming signal for emitting narrow band light having a luminance corresponding to the photometric signal.

The dimming unit 30 determines whether to end the processing (step S506). For example, when the doctor operates to stop the illumination light, the dimming unit 30 determines to end the processing. When the dimming unit 30 determines not to end the processing (NO in step S506), the dimming unit 30 returns to step S501. When the dimming unit 30 determines to end the processing (YES in step S506), the dimming unit 30 ends the processing.

According to the present embodiment, it is possible to provide the endoscope system 10 in which the automatic dimming function operates appropriately to prevent the output of the light source 28 from becoming too large even when the distal end of the endoscope 40 is close to or in contact with the biological tissue.

The operation of the dimming output unit 31 in the dimming unit 30 and the light source 28 described with reference to FIG. 3 is similar to an automatic dimming function used conventionally. Therefore, it is possible to realize the processor 20 for an endoscope in which the operation of the automatic dimming function is improved with a relatively small design change with respect to the conventional dimming unit 30.

### Second Embodiment

The present embodiment relates to an endoscope system 10 that determines whether there is a contact between a distal end portion 443 and a biological tissue based on an image of an end portion of an image sensor 46. The descriptions of portions common to those of the first embodiment will be omitted.

FIGS. 8A and 8B are explanatory diagrams for explaining a contact determination method according to a second embodiment. FIG. 8A schematically illustrates an endoscope image 61 when the distal end portion 443 is not in contact with the biological tissue. Black portions at the four corners indicate portions where so-called image vignetting occurs due to a mirror frame of the imaging optical system.

FIG. 8B schematically illustrates the endoscope image 61 when the distal end portion 443 is in contact with the biological tissue. Stray light due to, for example, refraction of light by the mucus is generated making upper right and lower right corner portions of the endoscope image 61 bright.

How the image vignetting occurs is easily affected by the variations in the assembly state between the image sensor 46 and the mirror frame. A control unit 21 displays, on a display device 16, the endoscope image 61 obtained by performing electronic mask processing on a region where the image vignetting may occur. Individual differences in the states of the four corners of the endoscope image 61 are prevented by the mask processing.

Even when the endoscope image 61 with bright corners is captured as illustrated in FIG. 8B, the control unit 21 displays the endoscope image 61 with black four corners on the display device 16. Therefore, the user, such as the doctor, does not notice the change in the brightness of the four corners.

The endoscope system 10 according to the present embodiment determines whether there is a contact between the distal end portion 443 and the biological tissue using data of the four corners on which the mask processing is normally performed.

FIG. 9 is a flowchart for explaining a processing flow of a program according to the second embodiment. The control unit 21 acquires an imaging signal from the image sensor 46 (step S601). The control unit 21 performs known signal processing on the imaging signal to generate the endoscope image 61 (step S602). The control unit 21 displays, on the display device 16, the endoscope image 61 on which the mask processing is performed on the four corners (step S603).

The control unit 21 starts a subroutine of contact determination (step S604). The subroutine of the contact determination is a subroutine that determines whether the distal end portion 443 and the biological tissue are in contact with each other. The processing flow of the subroutine of the contact determination will be described later.

The control unit 21 determines whether the contact has been determined by the subroutine of the contact determination (step S605). When the control unit 21 determines that there is no contact (NO in step S605), the control unit 21 instructs a dimming unit 30 to perform dimming (step S606). For example, the dimming unit 30 performs dimming for adjusting the brightness of a light source 28 based on the luminance of the B component of the imaging signal as conventionally performed. The dimming unit 30 may perform the operation similar to that in the first embodiment.

When the control unit 21 determines that there is a contact (YES in step S605), or after step S606 ends, the control unit 21 determines whether to end the processing (step S607). For example, when the doctor operates to stop the illumination light, the control unit 21 determines to end the processing. When the control unit 21 determines not to end the processing (NO in step S607), the control unit 21 returns to step S601. When the control unit 21 determines to end the processing (YES in step S607), the control unit 21 ends the processing.

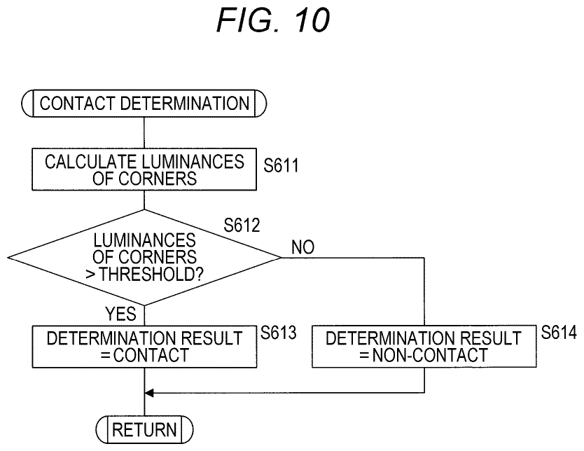

FIG. 10 is a flowchart for explaining a processing flow of the subroutine of the contact determination. The subroutine of the contact determination is a subroutine that determines whether the distal end portion 443 and the biological tissue are in contact with each other. The control unit 21 implements the function of a contact determination unit of the present embodiment by the subroutine of the contact determination.

The control unit 21 calculates the luminance of each of the four corner regions based on the endoscope image 61 before the mask processing is performed thereon (step S611). For example, the control unit 21 calculates an average value of the luminance of pixels in a region where vignetting always occurs regardless of variations in manufacturing an endoscope 40. The control unit 21 may calculate the luminance of the region where vignetting occurs in the endoscope image 61 at the time of white balance adjustment before the endoscopic examination is performed. The luminance may be, for example, a luminance(s) of one or two colors of R, G, and B. Instead of using the average value, an arbitrary statistical value such as a median value may be used.

The control unit 21 determines whether the luminance of each of the four corners exceeds a predetermined threshold (step S612). When the control unit 21 determines that the luminance of all corner portions exceeds the predetermined threshold (YES in step S612), the control unit 21 determines that a determination result of a contact state is “contact” (step S613). When the control unit 21 determines that there is a corner portion(s) that does not exceed the predetermined threshold (NO in step S612), the control unit 21 determines that the determination result of the contact state is “non-contact” (step S614). After step S613 or step S614 ends, the control unit 21 ends the processing.

Note that in step S612, the control unit 21 may determine whether the luminance of three or less corner portions exceeds the predetermined threshold. It is possible to realize the endoscope system 10 that detects contact even when only a very small portion of the distal end portion 443 is in contact with the biological tissue.

According to the present embodiment, since dimming is not performed when the distal end portion 443 and the biological tissue are in contact with each other, it is possible to provide the endoscope system 10 that prevents an increase in output of the light source 28 in a state where the endoscope image is darkened due to the contact.

Note that the dimming unit 30 may perform the contact determination described in the subroutine of the contact determination to determine whether to execute the dimming operation.

### Third Embodiment

The present embodiment relates to an endoscope system 10 capable of switching between a normal mode and a dark place mode suitable for capturing an image of a dark place. The descriptions of portions common to those of the second embodiment will be omitted.

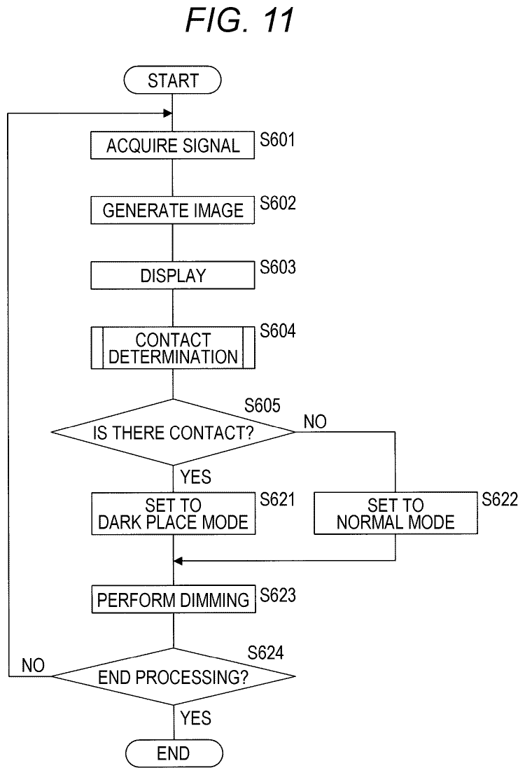

FIG. 11 is a flowchart for explaining a processing flow of a program according to a third embodiment. Since the processing from the start to step S605 is the same as the processing flow of the program of the second embodiment described with reference to FIG. 9, the description thereof will be omitted.

When the control unit 21 determines that there is a contact (YES in Step S605), the control unit 21 sets the endoscope system 10 to the dark place mode (step S621). The dark place mode is an operation mode suitable for observation in a dark place. When the control unit 21 determines that there is no contact (NO in step S605), the control unit 21 sets the endoscope system 10 to the normal mode (step S622). The normal mode is an operation mode suitable for normal endoscopic examination.

The difference between the dark place mode and the normal mode will be described. The dark place mode is, for example, a mode in which the analog gain of an image sensor 46 is higher than that in the normal mode, or a mode in which the speed of the electronic shutter is slower than that in the normal mode. The dark place mode may be a mode in which both the analog gain and the electronic shutter speed are changed from the normal mode. In steps S621 and S622, the control unit 21 implements the function of an element control unit that controls the setting of the image sensor 46.

Specific examples will be described. The analog gain in the normal mode is 1 time, and the analog gain in the dark place mode is 4 times larger than that in the normal mode. The speed of the electronic shutter in the normal mode is 1/240 seconds, and the speed of the electronic shutter in the dark place mode is 1/60 seconds which is slower than that in the normal mode.

After step S621 or step S622 ends, the control unit 21 instructs the dimming unit 30 to perform dimming (step S623). For example, the dimming unit 30 performs dimming for adjusting the brightness of a light source 28 based on the luminance of the B component of the imaging signal as conventionally performed. Note that, when the endoscope system 10 is set to the dark place mode, the dimming unit 30 outputs a dimming signal that makes the illumination light darker than that in the normal mode.

The control unit 21 determines whether to end the processing (step S624). For example, when the doctor operates to stop the illumination light, the control unit 21 determines to end the processing. When the control unit 21 determines not to end the processing (NO in step S624), the control unit 21 returns to step S601. When the control unit 21 determines to end the processing (YES in step S624), the control unit 21 ends the processing.

Note that, although the description of the flowchart is omitted, when the normal mode and the dark place mode are switched, it is desirable that the analog gain or the electronic shutter is gradually switched over a time of about 3 seconds. It is possible to provide the endoscope system 10 that prevents the user from feeling uncomfortable due to a sudden change in an endoscope image 61.

According to the present embodiment, when a distal end portion 443 and a biological tissue are in contact with each other, the endoscope system 10 is set to the dark place mode. In the dark place mode, the dimming unit 30 outputs a dimming signal that makes the illumination light darker than that in the normal mode, thus it is possible to provide the endoscope system 10 that prevents an increase in output of the light source 28 in a state where the endoscope image 61 is darkened due to the contact.

### Modification 3-1

Instead of starting the subroutine of the contact determination described with reference to FIG. 10, the control unit 21 may determine that the distal end portion 443 and the biological tissue are in contact with each other when PB is equal to or less than PG as described in Formula (5) of the first embodiment.

### Modification 3-2

The control unit 21 may gradually switch the endoscope system 10 to the dark place mode when PB/PG described in the first embodiment falls below 1, and may gradually switch the endoscope system 10 to the normal mode when PB/PG exceeds 2. It is possible to provide the endoscope system 10 that prevents frequent switching between the dark place mode and the normal mode in a state where it is not clear whether the distal end portion 443 and the biological tissue region comes into contact with each other continues.

### Fourth Embodiment

The present embodiment relates to an endoscope system 10 that changes components of illumination light when a distal end portion 443 and a biological tissue are in contact with each other. The descriptions of portions common to those of the third embodiment will be omitted.

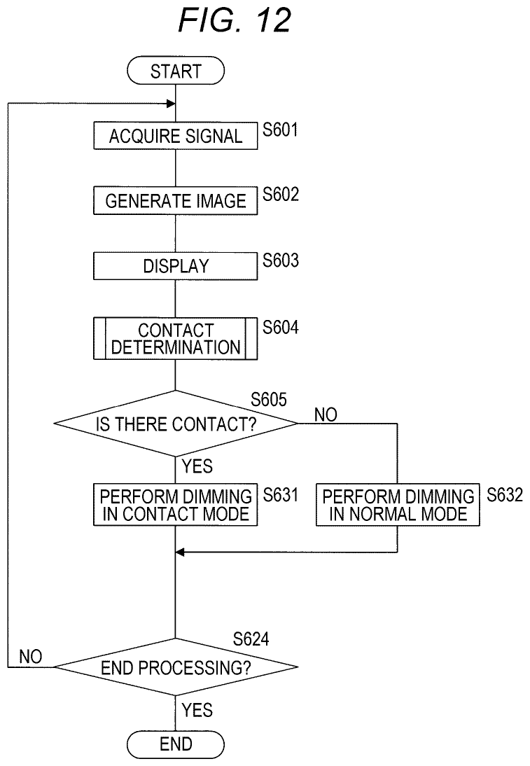

FIG. 12 is a flowchart for explaining a processing flow of a program according to a fourth embodiment. Since the processing from the start to step S605 is the same as the processing flow of the program of the second embodiment described with reference to FIG. 9, the description thereof will be omitted.

When the control unit 21 determines that there is a contact (YES in step S605), the control unit 21 instructs a dimming unit 30 to perform dimming in a contact mode (step S631). When the control unit 21 determines that there is no contact (NO in step S605), the control unit 21 instructs the control unit 21 to perform dimming in a normal mode (step S632).

After step S631 or step S632 ends, the control unit 21 determines whether to end the processing (step S624). When the control unit 21 determines not to end (NO in step S624), the control unit 21 returns to step S601. When the control unit 21 determines to end (YES in step S624), the control unit 21 ends the processing.

A difference between dimming in the contact mode and dimming in the normal mode will be described. The contact mode is a mode for causing a light source 28 to output illumination light having a smaller component of short-wavelength light than that in the normal mode. A case where special light observation is performed will be described as an example. The light source 28 in the normal mode emits light obtained by mixing violet light and green light at an energy ratio of 4:1. The light source 28 in the contact mode emits light obtained by mixing violet light and green light at an energy ratio of 2:1.

The illumination light obtained by mixing the violet light and the green light at the energy ratio of 4:1 is an example of illumination light having a first component ratio. The illumination light obtained by mixing the violet light and the green light at the energy ratio of 2:1 is an example of illumination light having a second component ratio in which the ratio of the violet light, which is the short-wavelength component, is lower than that in the first component ratio.

By reducing the ratio of violet light that is easily absorbed by the biological tissue, it is possible to suppress the reduction amount of light incident on the image sensor 46 even when the distal end portion 443 and the biological tissue are in contact with each other. Therefore, it is possible to provide the endoscope system 10 that prevents an increase in output of the light source 28 even when the distal end portion 443 and the biological tissue are in contact with each other.

### Modification 4-1

The control unit 21 may gradually switch the endoscope system 10 to the contact mode when PB/PG described in the first embodiment falls below 1, and may gradually switch the endoscope system 10 to the normal mode when PB/PG exceeds 2.

The technical features (constituent requirements) described in the embodiments can be combined with each other, and a new technical feature can be formed by the combination.

It should be noted that the embodiments disclosed herein are illustrative in all respects and are not restrictive. The scope of the present invention is defined not by the foregoing meanings but by the claims and is intended to include meanings equivalent to the claims and all modifications within the scope.

### REFERENCE SIGNS LIST

10 Endoscope system
16 Display device
17 Input device
20 Processor for an endoscope
21 Control unit
22 Main storage device
23 Auxiliary storage device
24 Communication unit
26 Display device I/F
27 Input device I/F
28 Light source
29 Connector for an endoscope
30 Dimming unit
31 Dimming output unit
32 Photometry unit
33 Luminance index acquisition unit
34 Generation unit
40 Endoscope
43 Operation unit
44 Insertion portion
441 Soft portion
442 Bending portion
443 Distal end portion
46 Image sensor
48 Scope connector
49 Universal cord
51 Observation window
52 Illumination window
53 Air supply nozzle
54 Water supply nozzle
55 Channel outlet
61 Endoscope image

1. A processor for an endoscope, comprising:
an imaging signal acquisition unit that acquires an imaging signal from an image sensor; and
a dimming unit that controls a light source that illuminates an observation visual field based on the imaging signal, wherein
the dimming unit includes:
a luminance index acquisition unit that acquires, based on the imaging signal, a first index related to a luminance of a first color and a second index related to a luminance of a second color;
a generation unit that generates a photometric signal based on the first index and the second index; and
a dimming output unit that outputs a dimming signal to the light source based on the photometric signal.

2. The processor for an endoscope according to claim 1, wherein
when the first index is smaller than the second index,
the generation unit generates the photometric signal on which a contribution degree of the second index is larger than that when the first index is larger than the second index.

3. The processor for an endoscope according to claim 1, wherein
when the first index is larger than the second index, the generation unit uses the first index as the photometric signal, and
when the first index is smaller than the second index, the generation unit generates the photometric signal larger than the first index.

4. The processor for an endoscope according to claim 1, further comprising
a contact determination unit that determines that a distal end of an endoscope is in contact with a biological tissue when the first index is smaller than the second index.

5. The processor for an endoscope according to claim 4, further comprising
an element control unit that controls setting of the image sensor, wherein
when the contact determination unit determines that there is no contact, the element control unit sets the image sensor to a normal mode used for normal image capturing, and
when the contact determination unit determines that there is a contact, the element control unit sets the image sensor to a dark place mode more suitable for capturing an image of a dark place than the normal mode.

6. The processor for an endoscope according to claim 1, further comprising
a contact determination unit that determines, based on the imaging signal, that a distal end of an endoscope is in contact with a biological tissue when a luminance of a corner portion of the image sensor is larger than a predetermined luminance.

7. The processor for an endoscope according to claim 6, further comprising
an element control unit that controls setting of the image sensor, wherein
when the contact determination unit determines that there is no contact, the element control unit sets the image sensor to a normal mode used for normal image capturing, and
when the contact determination unit determines that there is a contact, the element control unit sets the image sensor to a dark place mode more suitable for capturing an image of a dark place than the normal mode.

8. The processor for an endoscope according to claim 7, wherein
the dark place mode is a mode in which an analog gain is higher than that in the normal mode.

9. The processor for an endoscope according to claim 7, wherein
the dark place mode is a mode in which a shutter speed of an electronic shutter is slower than that in the normal mode.

10. The processor for an endoscope according to claim 4, wherein
when the contact determination unit determines that there is no contact, the generation unit uses the first index as the photometric signal, and
when the contact determination unit determines that there is a contact, the generation unit generates the photometric signal larger than the first index.

11. The processor for an endoscope according to claim 4, wherein
when the contact determination unit determines that there is no contact, the dimming unit outputs a dimming signal for causing illumination light having a predetermined first component ratio to be emitted, and
when the contact determination unit determines that there is a contact, the dimming unit outputs a dimming signal for causing illumination light having a second component ratio in which a ratio of a short-wavelength component is lower than the first component ratio to be emitted.

12. The processor for an endoscope according to claim 1, wherein
a wavelength of the first color is shorter than a wavelength of the second color.

13. The processor for an endoscope according to claim 12, wherein
the first color is blue, and
the second color is green.

14. A method of controlling a processor for an endoscope, executed by a control unit of the processor for an endoscope, comprising processing of:
acquiring an imaging signal from an image sensor;
acquiring, based on the imaging signal, a first index related to a luminance of a first color and a second index related to a luminance of a second color;
generating a photometric signal based on the first index and the second index;
outputting a dimming signal based on the photometric signal; and
controlling a light source that illuminates an observation visual field based on the dimming signal.
